(19) United States
(12) Patent Application Publication
(10) Pub. No.: US 2008/0085528 A1
(43) Pub. Date: Apr. 10, 2008

(54) GLUCOKINASE ACTIVITY ASSAYS FOR MEASURING KINETIC AND ACTIVATION PARAMETERS

(75) Inventors: Jovita Marcinkeviciene, Washington Crossing, PA (US); Kevin M. O'Malley, Chalfont, PA (US); Mark S. Kirby, New Hope, PA (US); Aiying Wang, West Windsor, NJ (US); Lisa M. Kopcho, Pemberton Borough, NJ (US); Ramakrishna Seethala, West Windsor, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/869,778

(22) Filed: Oct. 10, 2007

Related U.S. Application Data
(60) Provisional application No. 60/850,506, filed on Oct. 10, 2006.

(51) Int. Cl. C12Q 1/48; C12Q 1/54

(52) U.S. Cl. 435/14; 435/15

(57) ABSTRACT

The subject matter disclosed and claimed herein relates to novel in vitro assays for measuring glucokinase activity and use of these assays for identifying modulators of glucokinase.

This application claims priority to U.S. Provisional Application Ser. No. 60/850,506 filed on Oct. 10, 2006.

The subject matter disclosed and claimed herein relates to assays and methods for evaluating glucokinase (“GK”) activity by directly measuring product(s) produced from GK enzymatic reactions. Such reaction products include adenosine di-phosphate (“ADP”) and/or glucose-6-phosphate (“G-6-P”). A distinguishing feature of the methods disclosed herein is the ability to accurately measure maximal GK activation and, as a consequence, the ability to more accurately determine the concentration of agents required to achieve half maximal activation of GK (AC₅₀). Unlike previously reported assays, the assays disclosed herein are not affected by coupling reagents. As a result, there is an increase in accuracy of the assays, and the disclosed assays provide a substantial improvement for measuring GK activity. Moreover, the assays disclosed herein can be readily adapted for medium to high throughput screening of agents that modulate GK activity.

### BACKGROUND OF THE INVENTION

Glucokinase is a hexokinase family member and catalyzes the first step in glycolysis. GK is one of the four mammalian glucose phosphorylating isoenzymes and serves as a glucose sensor in specific tissues requiring “glucose sensing”, such as the liver, pancreatic β-cells, hypothalamus, pituitary, and K- and L-enteroendocrine cells of the GI tract. (Matschinsky, F. M. (1990). “Glucokinase as glucose sensor and metabolic signal generator in pancreatic b cells and hepatocytes.” Diabetes 39: 647-652). Unlike other hexokinase family members, GK has a distinctive structure, enzymatic activity and tissue localization. GK has a higher Kₘ than the other hexokinases for glucose over the typical physiological range (about 3 to 12 mM). Kₘ is typically defined as a parameter which is indicative of substrate concentration at which half of the maximal velocity of an enzymatic reaction is achieved.

GK is the only family member known to have an allosteric activation site. Activation of GK by overexpression, genetic mutations, and small molecule allosteric activators have all been shown to increase insulin secretion and decrease whole body glucose load. This activity suggests agents that bind to this allosteric site, and activate GK, could serve as specific anti-diabetic agents.

GK's significant role in the control of blood glucose levels is underscored by research using transgenic animals and in humans possessing GK mutations. For example, pancreatic or liver-specific GK knockout mice display hyperglycemia (Postic, C., et. al., (1999). “Dual roles for glucokinase in glucose homeostasis as determined by liver and pancreatic b cell specific gene knockouts using Cre recombinase.” J. Biol. Chem. 274: 305-315.). Overexpression of GK leads to lower fasting blood glucose levels and resistance to the development of high fat diet-induced diabetes (Niswender, K. D., et. al., (1997). “Cell-specific expression and regulation of a glucokinase gene locus transgene.” J. Biol. Chem. 272: 22564-22569; Shiota, M., et al., (2001). “Glucokinase gene locus transgenic mice are resistant to the development of obesity-induced type 2 diabetes.” Diabetes 50: 622-629). In humans, naturally occurring inactivating and activating mutations in the gene encoding GK were reported to cause maturity onset diabetes of the young type-2 (MODY2) (Vionnet, N., et. al., (1992). “Nonsense mutation in the glucokinase gene causes early onset non-insulin-dependent diabetes mellitus.” Nature 356: 721-722; Froguel, P., et. al., (1993). “Familial hyperglycemia due to mutations in glucokinase. Definition of subtype of diabetes mellitus.” N. Engl. J. Med. 328: 697-702.), and persistent hyperinsulinemic hypoglycemia of infancy (PHHI) (Glasser, B., et al., (1998). “Familial hyperinsulinism caused by an activating glucokinase mutation.” N. Engl. J. Med. 338: 226-230).

The data reported in the literature reflect the importance of GK in regulating glucose homeostasis and suggest that pharmacological modulation (e.g., activation) of GK in patients having diabetes-related disorders could have therapeutic benefits. In recent years, several groups reported discovery of small molecules that enhance GK activity by apparent binding to the GK allosteric site. The identified compounds stimulate insulin secretion in a glucose-dependent manner in pancreatic B-cells and increase glucose uptake in rat hepatocytes. Additionally, it was observed that GK activators lowered blood glucose levels and improved glucose tolerance tests in wild type and Diet Induced Obese mice (DIO) (Grimsby, J., et. al., (2003). “Allosteric Activators of Glucokinase: Potential Role in Diabetes Therapy.” Science 301: 370-373; Efanov, A. M., et. al., (2005). “A Novel Glucokinase Activator Modulates Pancreatic Islet and Hepatocyte Function.” Endocrinology 146: 3696-3701; McKerrecher, D., et. al., (2006). “Design of a potent, soluble glucokinase activator with excellent in vivo efficacy.” Bioorg. Med. Chem. Letters 16: 2705-2709).

Despite the progress in identifying modulators of GK, reliable in vitro assays and models have yet to be fully developed to accurately and reproducibly evaluate GK modulators. In the past, enzyme activation assays involved measurement of reaction velocity based on (thio)-NADH generation, in the presence of glucose-6-phosphate dehydrogenase (GDH), to monitor production of glucose-6-phosphate. (Castelhano, A. L., et. al. (2005). “Glucokinase activating ureas.” Bioorg. Med. Chem. Letters 15: 1501-1504). In such assays, GK, glucose, potential GK modulators, Thio-NADH, and GDH were all contained in the same reaction vessel. However, due to 1) the presence of ATP in the assay: 2) the inhibitory effect that relatively high glucose concentration has on GDH activity; and 3) the different sugar anomeric preference shown by GK, the data obtained in previously described GK measurement assays does not necessarily reflect of GK activity alone. (Cleland W. W. (1979). “Optimizing Coupled Enzyme Assays.” Analytical Biochem. 99: 142-145.; Malaisse, W., et. al., (1985). “Anomeric specificity of glucose metabolism in pentose cycle.” J. Biol. Chem. 260: 14630-14632; Curtois, P. et. al., (2000). “Anomeric specificity of human liver and b-cell glucokinase: modulation by the glucokinase regulatory protein.” Arch. Biochem. Biophys. 373: 126-134.).

In view of the shortcomings of known GK-related assays, the subject matter disclosed and claimed herein provides an improvement for measuring GK activity by direct measurement of GK product formation using: a) high performance liquid chromatography (“HPLC”) measurement of ADP; b) direct G-6-P measurement by ion-exchange filtration; and/or c) a tandem assay that facilitates uncoupled detection of glucose-6-phosphate.

These assays represent a marked improvement over what has been previously described and provide accurate and reproducible in vitro assays for measuring GK activity. These assays facilitate screening (low, medium, and high throughput) for modulators of GK. Given the physiological role played by GK, identification of GK modulators (e.g., activators) should have several beneficial actions including: decreasing hepatic glucose output; increasing hepatic glucose disposal; increasing pancreatic insulin secretion by direct actions and indirect (incretin) effects; increasing incretin release from K- and L-enteroendocrine cells; and possible effects on feeding and energy homeostasis in the ventromedial hypothalamus (VMH).

### SUMMARY OF THE INVENTION

Briefly, there are three assays described herein that provide accurate and reliable methods for measuring GK activity.

The first assay is an HPLC-based assay that features measurement of ADP produced from the GK reaction. The HPLC-based assay involves a method for measuring glucokinase (GK) activity comprising: incubating GK with substrate; initiating a GK reaction; stopping the reaction; and determining adenosine di-phosphate (ADP) concentration using high performance liquid chromatography (HPLC). The HPLC-based assay may further comprise the following: substrates comprising glucose and adenosine tri-phosphate (ATP); and the GK reaction has a higher maximal (activation) activity than observed in a coupled GK assay. The HPLC assay may be used to screen for modulators of GK. Such methods comprise: incubating GK with substrate in a control reaction vessel; incubating GK with substrate and a test compound in a test reaction vessel; initiating a GK reaction in each reaction vessel; stopping the reaction; determining ADP concentration in each reaction vessel using HPLC; and comparing ADP concentration from the control and test reaction vessels, wherein a higher ADP concentration in the test reaction vessel, relative to the control reaction vessel, reflects that the test compound is a GK activator. The screening methods may further comprise the following: substrates (e.g., glucose and ATP); and the test compound is delivered in dimethysulfoxide (DMSO) or another suitable carrier.

Another assay disclosed herein is a filtration based assay wherein radiolabeled G-6-P is measured using a scintillation counter and the relative concentration of G-6-P is determined. The filtration based assay involves a method for measuring GK activity comprising: incubating GK with substrate and reagents, wherein the reagents comprise radiolabeled glucose; initiating a GK reaction to yield reaction products; stopping the reaction; filtering the reaction products; washing the reaction products; eluting the reaction products; and determining glucose-6-phosphate concentration by quantification of radiolabeled glucose-6-phosphate produced by the GK reaction.

The filtration based assay may further comprise tritiated glucose as the radiolabeled glucose; glucose in a range of about 0.33 mM to about 50 mM; initiating the reaction by addition of Mg-ATP; stopping the reaction by addition of formic acid; and washing the reaction products with water.

The filtration based assay may also be used to screen for GK modulators. A method for screening for GK modulators includes: incubating GK with substrate and reagents, wherein the reagents comprise radiolabeled glucose, in a control reaction vessel; incubating GK with substrate, reagents, and a test compound, wherein the reagents comprise radiolabeled glucose, in a test reaction vessel; initiating a GK reaction to yield reaction products in each vessel; stopping the reaction; filtering the reaction products; washing the reaction products; eluting the reaction products; determining glucose-6-phosphate concentration by quantification of radiolabeled glucose-6-phosphate produced by the GK reaction; and comparing glucose-6-phosphate concentration from the control and test reaction vessels, wherein a higher glucose-6-phosphate concentration in the test reaction vessel, relative to the control reaction vessel, reflects that the test compound is a GK activator.

The screening method using the filtration based assay may further comprise: conducting the method is conducted in a high-throughput format; use of tritiated glucose; use of glucose in a range of about 0.33 mM to about 50 mM; initiating the reaction by addition of Mg-ATP; terminating the reaction by addition of formic acid; washing the reaction products with water; and/or delivering a test compound in DMSO.

Finally, described and claimed herein is a tandem assay wherein G-6-P is measured using absorbance and the relative concentration of G-6-P is determined. One of the benefits of the tandem assay is avoidance of the technical difficulties often associated with conventional coupling assays, wherein components from the second ‘coupling’ interfere with the GK reaction.

The tandem assay involves use of a two step process wherein the offending reagents used in known coupled assays, that may otherwise impact the data obtained from the assay, are kept separate thereby facilitating a more accurate GK measurement. For example following an initial reaction with GK, glucose, and ATP, the reaction is stopped (by heating or quenching with ethylenediaminetetraacetic acid “EDTA”) and the reaction mixture transferred to a fresh microtiter plate. ThioNAD and glucose-6-phosphate dehydrogenase (G-6-PDH) are then added to the fresh plate and reacted—termed a “second reaction.” Reagents used in this second reaction that typically interfere with GK enzymatic activity, are not present during the initial reaction. The plate contents are then mixed well, and this second reaction is incubated for a sufficient period of time to allow the GK reaction to run to completion. Following the incubation period, the absorbance of each well is read using a plate reader.

The tandem assay for measuring GK activity involves a method including: incubating GK with glucose in a first reaction vessel; adding Mg-ATP to the first reaction vessel to initiate a GK enzymatic reaction and thereby form a reaction mixture; stopping the GK enzymatic reaction; transferring the reaction mixture to a second reaction vessel; adding Thio-NAD and glucose-6-phosphate dehydrogenase to the reaction mixture in the second reaction vessel; mixing the contents of the second reaction vessel; and determining glucose-6-phosphate concentration by measurement of the absorbance of contents (thio-NADH produced during G-6-PDH the reaction) of the second reaction vessel and correlating absorbance values to a glucose-6-phosphate standard curve. The methods associated with the tandem assay further comprises: conducting the reaction in a multi-well microtiter plate; use of glucose over a range of concentrations from 0 to about 50 mM; and cooling of the reaction mixture on ice prior to transfer to the second reaction vessel.

The tandem assay may be used to screen for modulators of GK. Such an assay involves a method for screening for modulators of GK activity comprising: incubating GK with glucose in a first control reaction vessel; incubating GK with glucose and a test compound in a first test reaction vessel; adding Mg-ATP to the first control and test reaction vessels to initiate GK enzymatic reactions and thereby form reaction mixtures; stopping said GK enzymatic reactions; transferring aliquots of the reaction mixtures to a second control and second test reaction vessel; adding Thio-NAD and glucose-6-phosphate dehydrogenase to the reaction mixture in the second control and test reaction vessels; mixing the contents of the second control and test reaction vessels; determining glucose-6-phosphate concentration by measurement of the absorbance of the contents (thio-NADH) of the second control and test reaction vessels; correlating absorbance values obtained from the second control and test reaction vessels, to a glucose-6-phosphate standard curve; and comparing glucose-6-phosphate concentration from the second control and test reaction vessels, wherein a higher glucose-6-phosphate concentration in the second test reaction vessel, relative to the second control reaction vessel, reflects that said test compound is a GK activator.

The method for screening for GK modulators may further comprise: conducting the reactions in multi-well microtiter plates; use of glucose over a range of concentrations from 0 to about 50 mM; and cooling of the first control and test reaction vessels on ice prior to transfer to the second control and test reaction vessels.

### DETAILED DESCRIPTION OF THE INVENTION

### Definitions & Abbreviations

The abbreviations, terms, and phrases used herein are defined as follows.

The term “Kₘ” is defined as the substrate concentration at which half of the maximal enzymatic reaction rate is achieved.

The term “k_{cat}” is defined as the maximal reaction rate at saturating substrate(s) concentration per enzyme concentration.

The phrase “maximal activation” is defined as the maximum activity observed for an enzyme with a sufficient amount of substrate present and available and at the saturating levels of the activator.

The term “AC₅₀” is defined as the concentration of agent required to achieve half-maximal activation of enzymatic activity (e.g., GK activity).

The term “modulator” is defined as an agent that is capable of altering the activity of a target such as an enzyme. A modulator may be an activator or an inhibitor and may comprise small chemical molecules, biologics (e.g., antibodies, antibody fragments, domain antibodies, peptide binding agents, etc.), nucleic acids (e.g., DNA, RNA, cDNA), amino acids, and/or polypeptides.

The term “activator” is defined as an agent that causes increased activity of a target, such as an enzyme. An activator of GK would increase GK activity and likely lead to an increase in product formed by GK and/or consumption of substrate used by GK.

The term “about” when used to describe numerical ranges includes those values which are +/−20% of the recited or described value.

The term “high-throughput” is defined, in the context of an assay, as allowing multiple test agents to be screened for binding and/or activity of a target (e.g., GK)

### BRIEF DESCRIPTION OF THE FIGURES

FIG. 1 reflects data corresponding to maximal activation of GK by (R)-diethyl (5-(3-cyclopentyl-2-(4-(methylsulfonyl)phenyl)propanamido) pyrazin-2-yl)methylphosphonate (Compound A) at 5 mM glucose when measured using HPLC and coupled assays.

FIG. 2 reflects data corresponding to maximal activation of GK by Compound A at 20 mM glucose when measured using HPLC and coupled assays.

FIG. 3 reflects comparative AC₅₀ data measured by HPLC and coupled assays using Compound A.

FIG. 4 reflects the different effects produced by (R)-diethyl(2-(3-cyclopentyl-2-(4-(methylsulfonyl)phenyl)propanamido)thiazol-5-ylthio)methylphosphonate (Compound B) on k_{cat} and Kₘ of GK when measured using a coupled assay and a direct HPLC assay.

FIG. 5 reflects the separation of glucose-6-phosphate (product) from glucose (substrate) by filtration using an ion-exchange resin.

FIG. 6 reflects the Kₘ value for glucose in the presence and absence of an activator by filtration assay.

FIG. 7 reflects data comprising a dose-response curve for an activator on GK activity in the presence of 12 mM glucose in a filtration assay.

FIG. 8 reflects the K_{M} value for glucose in the presence and absence of a GK activator using a tandem assay.

FIG. 9 reflects data comprising a dose-response for a GK activator (Compound C) of human hepatic GK activity in the presence of 12 mM glucose in a tandem assay.

FIG. 10 reflects data comprising a dose response for a GK activator of human hepatic GK activity in the presence of 12 mM glucose in a tandem assay with EDTA Quench.

The following embodiments are illustrative of the subject matter disclosed and claimed herein. At the time of filing, one of ordinary skill in the art would have understood that variations of the described embodiments are contemplated and embraced by the specification and claims provided herein.

### EXAMPLE 1

### HPLC-Based ADP Detection as a Measure Of GK Activity

As described above, GK, when activated, produces ADP if effective concentrations of glucose and ATP are available for use by GK to initiate the steps in glucose metabolism. The HPLC-based assay involves the measurement of ADP production by GK.

Human full-length recombinant GK was used to measure the activation parameters of GK in an HPLC-based assay. Recombinant GK (15 nM), along with reaction solutions containing 25 mM Hepes (pH 7.1), 1 mM DTT (freshly added daily), and various concentrations of glucose were mixed with 5 mM ATP containing 6 mM MgCl₂ (pH adjusted) to initiate the GK reaction in Eppendorf tubes. GK modulators (e.g., activators such as Compound A) were introduced as 100% DMSO stock solutions and the final DMSO concentration was 5%. When GK modulators were added to the reaction mixtures, appropriate controls using the DMSO vehicle were included.

To establish a time course for the in vitro GK reaction, reaction mixtures were incubated at room temperature and periodically quenched by boiling for 1 minute. After mixing (1:1) the reaction mixtures with HPLC mobile phase buffer (Buffer A: 35 mM KH₂PO₄ with 6 mM tetrabutyl ammonium hydrogen sulfate, and 12.5 mM EDTA, pH 6.0) samples were injected onto a YMC Hydrosphere C-18 Column (Waters, Size=150×4.6 mm, Particle=S-3 μM). ADP and ATP peaks were resolved using a modified reverse-phase protocol (Pietta, P., et. al., (1987). “High-performance liquid chromatographic assay for hexokinase.” Journal of Chromatography 390: 458-462; Horiuchi, K. Y., et. al., (2001). “Mechanistic studies of reaction coupling in Glu-tRNA amidotransferase.” Biochemistry 40: 6450-6457).

Note that commercial samples of ATP often have an approximate 1-2% ADP contamination. As such, there is a possibility of observing a background ADP peak. Any background peaks were monitored closely and subtracted from all ADP peaks identified following the reaction. Peak areas were converted to ADP concentrations using a calibration curve. In all circumstances, including those where GK were added to the reaction, the conversion (by ATP) did not exceed 8%.

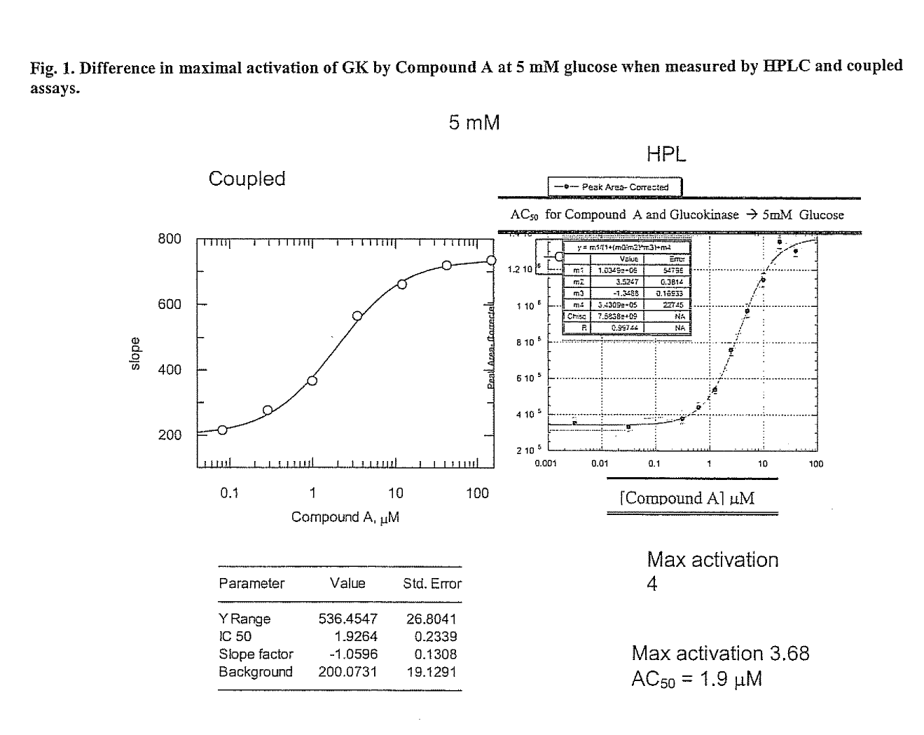

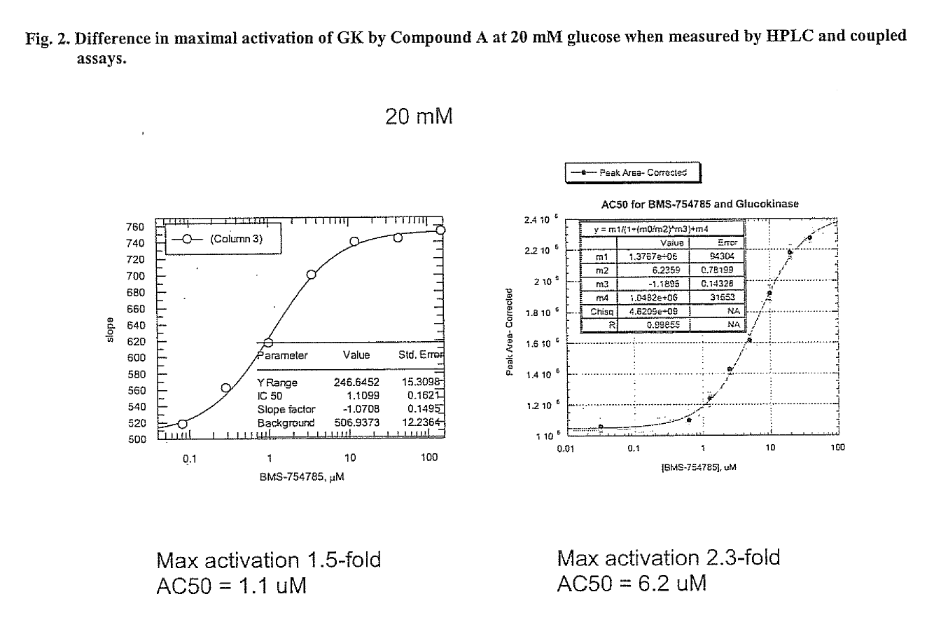

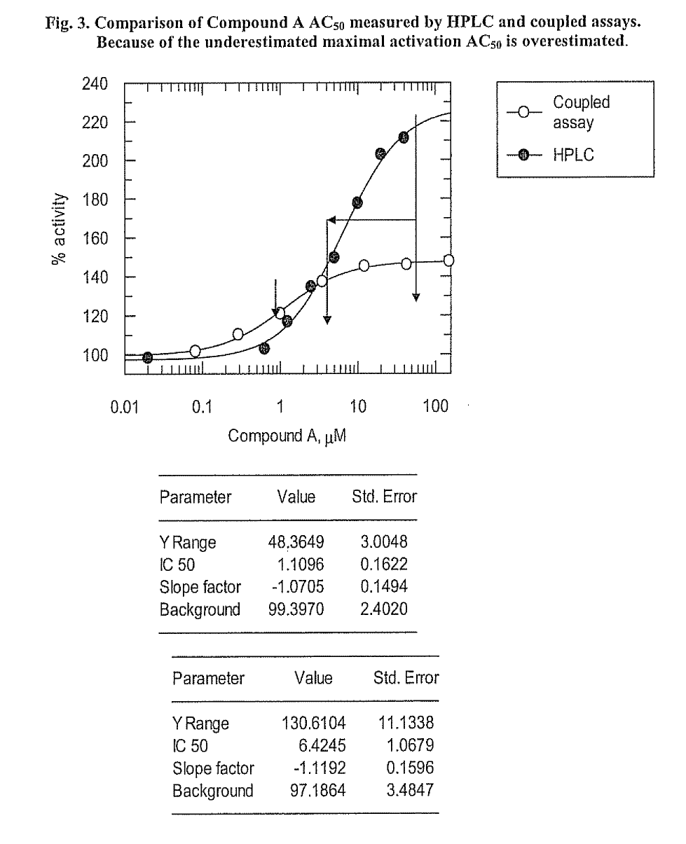

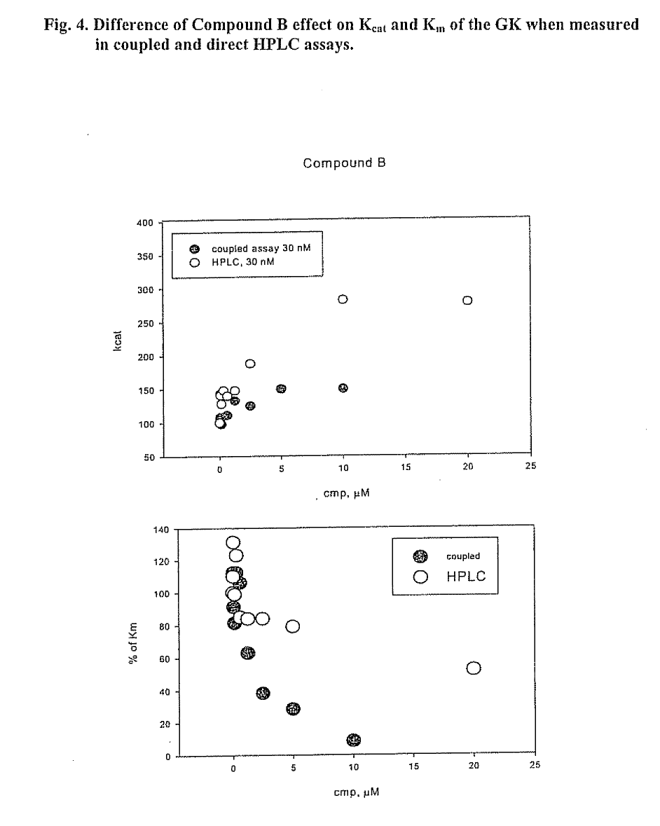

Data for individual compound concentrations were fit to a 4-parameter equation (using Grafit® software) to calculate AC₅₀ (concentration at which half maximal activation is achieved) and maximal activation (Yₘₐₓ):

$$y = \frac{{Y}_{max} - {Y}_{0}}{1 + {( \frac{A}{A {C}_{50}} )}^{n}} + {Y}_{0}$$

where Y₀ is background (which usually is equal to the control, non-activated reaction); A is the activator concentration and n is slope of the curve. The GK activation parameters of Compound A tested were measured using the above-described HPLC-based activity assay and compared with those obtained using a coupled assay (i.e., coupled to G-6-P dehydrogenase continuous assay). The data are reported in FIGS. 1-4.

The coupled assay is typically conducted in a 96-well microtiter plate. The reaction buffer (25 mM Hepes, pH 7.1 with 1 mM DTT and 6 mM MgCl) is mixed with GK (50 nM), a test compound (e.g., Compound A) and appropriate concentration(s) of glucose. Following mixing of these reagents, coupling reagents (1 mM Thio-NAD+ and 20 U/ml G6PDH) are added and the reaction is initiated by addition of 5 mM ATP. Reaction progress is monitored by measuring the appearance of Thio NADH at 405 nm using a spectrophotometer. Note that ADP production was correlated with G-6-P generation during the GK reaction in the presence of the activator to ensure that activator did not “uncouple” the reaction and produce ADP without generating G-6-P.

The data reflected in FIGS. 1-4, using the HPLC-based assay, indicates that prior to development of the assay(s) disclosed herein, the maximal activation of GK was underestimated. One possible reasons for the underestimation include that if the rate of the activated reaction was limited by the coupling reaction. That is, there may not have been a sufficient amount of coupling enzyme in the reaction mixture (when the reaction is activated) or the coupling enzyme activity may have been inhibited by the ADP. Additionally, beta-glucose is a preferred substrate for G6PDH and this preference may contribute to the rate underestimation under the initial velocity conditions since GK phosphorylates both alpha and beta glucose.

Because of this underestimation, the AC₅₀ for activators of GK was overestimated. The assays described herein demonstrate that GK activators produce a much higher maximum activation level than previously understood, and, as a result, requires a higher concentration of GK activator to reach half maximal activation (i.e., has a higher AC₅₀ value). This discovery provides a more accurate model of GK activity and will lead to generation of more reliable data for evaluating modulators of GK activity.

### EXAMPLE 2

### Direct Glucose-6-Phosphate Capture by Filtration Using an Ion-Exchange Resin

In addition to the HPLC-based ADP measurement assay described in Example 1, disclosed herein is a filtration based assay for measurement of the amount of G-6-P produced by activated GK.

Human full-length recombinant GK was used to measure the activation of GK. Human full-length GK (15 nM) was incubated with various concentrations of glucose in the range from 0.33 to 50 mM in the presence of tritiated glucose (3H-glucose [6-³H], 0.33 μCi) in 96 well microtiter plates. To initiate the GK reaction, Mg-ATP (3 mM final) was added to the protein in buffer, under the final buffer conditions of 25 mM HEPES, pH 7.1, containing 1 mM DTT and 5% DMSO. The total reaction volume was 110 μl. The reaction was allowed to proceed for ten minutes (i.e., the linear portion of the reaction) and was then quenched with 100 mM formic acid (1:1). A 200 μl aliquot of the quenched reaction products was then transferred to wells in a 96-well MultiScreen-GV 96 filtration plate containing 100 μl/well of Bio-rad AG 1-X8 Resin, formate form. The resin was then washed with H₂O (1 ml/well), and the GK reaction product (G-6-P) eluted with 1M ammonium formate, pH 5 (200 μl/well). A 50 μl aliquot of this eluate was added to an Optiplate white 96 well plate containing 200 μl of Microscint PS. The plate was sealed and shaken for 5 minutes, and read on a Topcount scintillation counter (Perkin Elmer). The raw counts were converted to product concentration by comparison of the raw counts in the sample wells to wells containing a standard curve having known concentrations of ³H-glucose-6-phosphate.

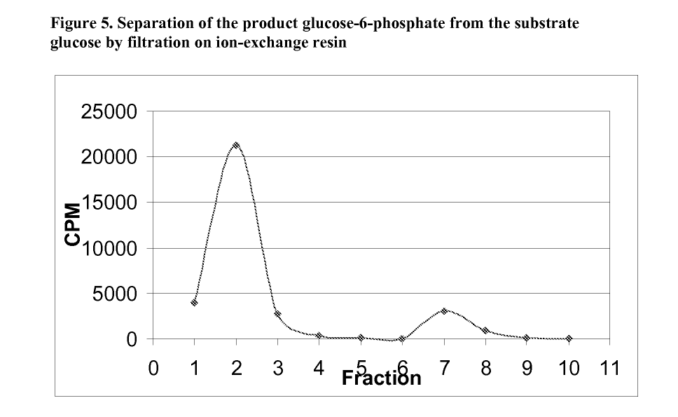

The data reported in FIGS. 5 through 7 correspond to work conducted using the filtration-based assay. FIG. 5 reflects the elution pattern of G-6-P following washing of the resin with H₂O to remove the substrate glucose, followed by elution of the product G-6-P with ammonium formate. A separation of substrate versus converted product is seen, as the peak due to ³H-glucose diminishes to background following the water wash, and a secondary peak due to ³H-glucose-6-phosphate elutes following addition of ammonium formate. These data support the conclusion that the filtration based assay can be successfully used to separate glucose from the G-6-P product.

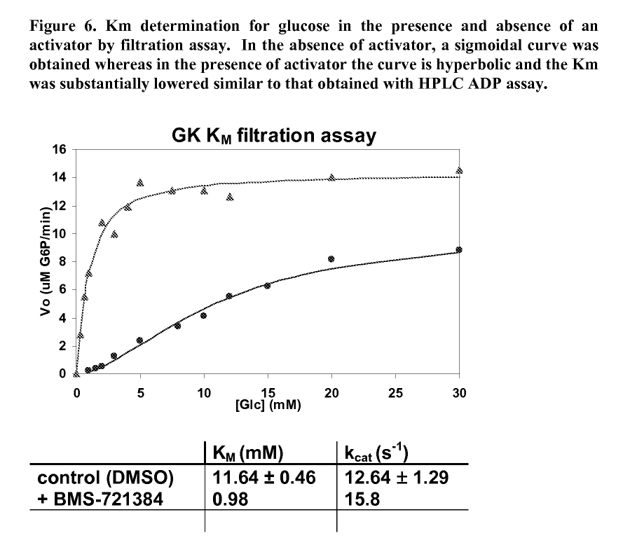

FIG. 6 reflects the GK kinetic parameters observed using the filtration-based assay. The protocol used is essentially as described above (Example 2) except the recombinant GK was incubated with various concentrations of glucose in the range from 0.33 to 50 mM in the presence of tritiated glucose both in the presence (triangles in FIG. 6) and absence (circles in FIG. 6) of a GK activator. FIG. 6 shows the characteristic sigmoidal curve of the GK enzymatic reaction with increasing amounts of glucose (circles). In the presence of 20 PM of activating compound (triangles), the curve loses a sigmoidal characteristics and changes to a hyperbolic curve, which is consistent with response data reported in the literature for a coupled assay (Grimsby et al., 2003, supra).

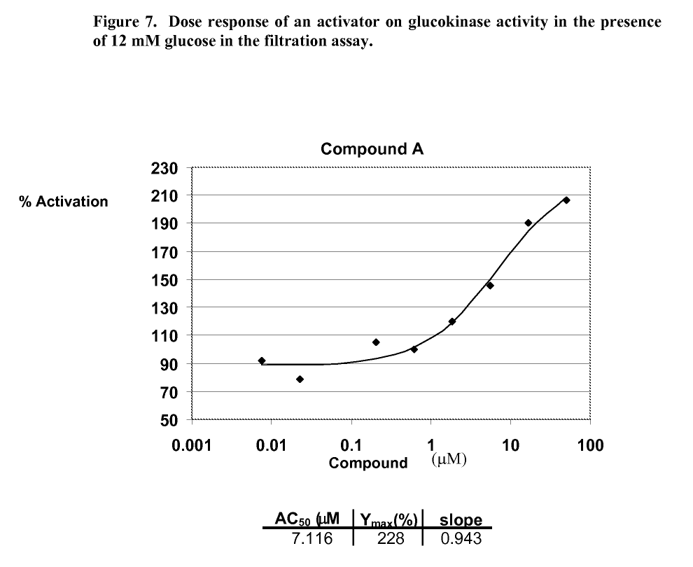

FIG. 7 provides activation and AC₅₀ data obtained using the filtration-based assay which are similar to those obtained using the HPLC-ADP assay described in Example 1 (compare FIGS. 1-4 and FIGS. 5-7. For example, FIG. 7 shows the effect of Compound A on the activity of human hepatic GK. Compound A activates GK with a maximal activation number of 228% (above background) and a corresponding AC₅₀ value of 7.1 μM.

### EXAMPLE 3

### Uncoupled Detection of G-6-P Using a Tandem Assay

A third assay described herein useful for measuring GK activity is a tandem assay. A particularly useful feature of the tandem assay is the ability to use the assay in a high throughput screen for GK modulators.

Purified human recombinant GK was used to measure the activation of GK activity by glucose and a GK activator (Compound “C”) in the tandem assay. A suitable vehicle (for instance 24% DMSO in pH 7.25 Tris buffer), with or without (control) a GK modulator of interest was incubated with GK (50 μl of a 24 nM stock solution) over a range of concentrations of glucose (for instance from 0.32 to 80 mM) for 30 minutes in a 96-well PCR plate (10 μl). The GK reaction was initiated by addition of Mg-ATP (20 μl). The solution used comprised 12 mM ATP and 16 mM MgCl₂. The final assay conditions were 25 mM Tris, pH 7.25, 1 mM DTT and 3% DMSO, 15 nM GK, 3 mM ATP and 4 mM MgCl₂. Glucose stock solution (IM) was diluted to generate a dilution series with final glucose concentrations of: 0.2, 0.4, 0.6, 0.8, 1, 1.5, 2, 3, 4, 5, 6, 8, 10, 12, 20 and 50 mM. The total reaction volume per well was about 80 μl.

The GK reaction proceeded for about 10 minutes, and was followed by heating the 96-well plate in a hot water bath (100° C.) for 30 seconds to stop the reaction. The assay plate was then cooled on ice, and centrifuged at 1000 rpm for 1 minute. An aliquot of reaction mixture (typically 50 μl) was then transferred to a second 96 well plate suitable for taking absorbance measurements. The second plate contained 4 mM ThioNAD and 20 Unit/ml of G-6-P-DH (100 μl). The reagents and reaction mixture are mixed for 2 minutes, followed by measurement of the absorbance at 405 nm using a plate reader. Note that the assay plate(s) contained wells having 6 to 10 dilutions of glucose-6-phosphate concentration, typically ranging from 10 μM to 1 mM, to allow generation of a standard curve. The amount of glucose-6-phosphate for the samples of interest were then obtained from this standard curve of glucose-6 phosphate.

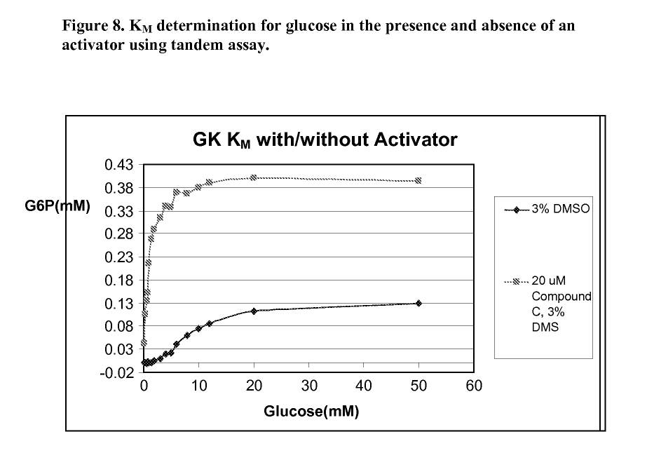

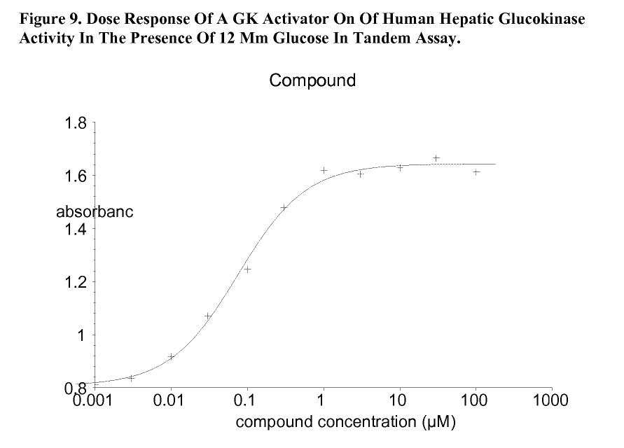

FIGS. 8 and 9 reflect data corresponding to the tandem assay to determine Kₘ and k_{cat} (FIG. 8) and GK activation by a test compound (FIG. 9).

The kinetic parameters of GK, as measured using the tandem assay, were validated. Data corresponding to this work is described in FIG. 8. The data corresponding to FIG. 8 was generated using the protocol generally described above, and specifically described as follows. As shown in FIG. 8, in the absence of activator, a sigmoidal curve was observed. In the presence of the activator compound A, the curve is hyperbolic and the K_{M} was substantially lower compared to the controls. The data obtained using the tandem assay is similar to the data obtained using the direct HPLC (Example 1) and Filtration (Example 2) assays described herein. Signal produced by stopping the reaction with EDTA was stable up to 1 hour.

A variation of the uncoupled assay is further provided. In the variation EDTA is employed to stop the reaction rather than the use of a heating step and a 3 84 well format is used. The use of EDTA quenching facilitates the GK enzymatic reaction, quenching, and spectral observation in the same well of a microplate.

The protocol used is essentially as described above with some variation. Briefly, human full-length GK (15 nM) was incubated with various concentrations of glucose in the range from 0.33 to 50 mM in clear bottom 384 well microtiter plates. To initiate the GK reaction, Mg-ATP (3 mM final concentration) was added to the protein in buffer, under the final buffer conditions of 25 mM HEPES, pH 7.1, containing 1 mM DTT and 5% DMSO. The total reaction volume was 20 μl. The reaction was allowed to proceed for ten minutes and was then quenched with 5 μl EDTA (45 mM final).

The components of the secondary reaction, ThioNAD and G6PDH (final concentrations of 650 μM and 3.33 Units, respectively), were then added together in a volume of 25 μl, and a total volume of 50 μl. Absorbance was read and activation calculated as a percentage of background activity, i.e., GK in the presence of DMSO, with background G6P subtracted. Background G6P was determined by pre-quenching GK with EDTA prior to reaction initiation with ATP.

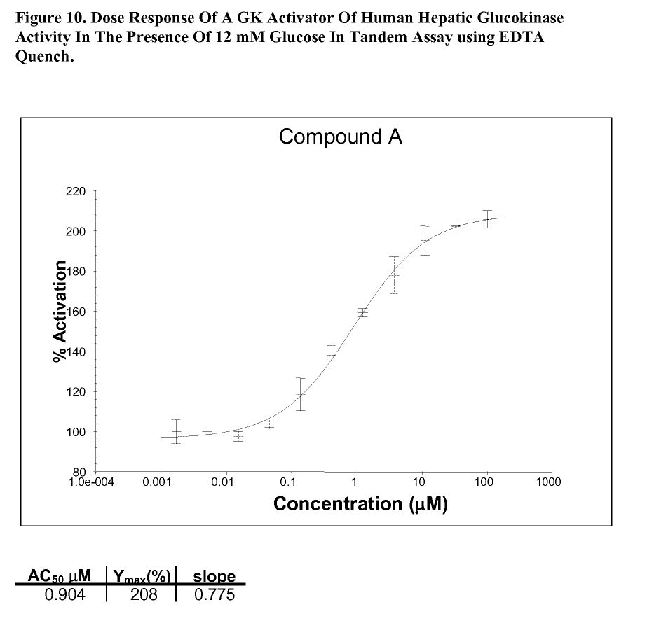

The tandem assay (EDTA quench) was used to assess the activity of GK in the presence of putative GK activator compounds. The above-described tandem assay protocol was followed using a range of activator compound concentrations from 0 to 100 μM. A representative response curve obtained using 12 mM glucose is reported in FIG. 10. These data reflect that Compound A activates GK with an AC₅₀ value of about 0.904 μM and a maximal activation of 208% of background GK activity. These data are consistent with data obtained using the other HPLC (Example 1) and Filtration (Example 2) assays as well as Tandem assay format with heat quench as described herein.

TABLE 1
K_{M} and k_{cat} For Glucose In The Presence And
Absence Of A GK Activator In A Tandem Assay.

| Agent Added | K_{M}(mM) | K_{cat}(S⁻¹) |
|---|---|---|
| Control(3% DMSO) | 8.90 ± 0.14 | 15 |
| 20 uM Compound A | 0.97 ± 0.01 | 44 |

The tandem assay was used to assess the activity of GK in the presence of putative GK activator compounds. The data corresponding to this work is reported in FIG. 9. The tandem assay protocol followed was as described above using a range of activator compound from 0 to 100 μM. A representative response curve obtained using 12 mM glucose is reported in FIG. 9. The data reflect that Compound C activates GK with an AC₅₀ value of about 0.075 μM and a maximal activation of 204% of background GK activity. The data are consistent with data obtained using the other HPLC (Example 1) and Filtration (Example 2) assays described herein.

What is claimed is:

1. A method for measuring glucokinase (GK) activity comprising:
a. incubating GK with substrate;
b. initiating a GK reaction;
c. stopping said reaction; and
d. determining adenosine di-phosphate (ADP) concentration using high performance liquid chromatography (HPLC).

2. The method of claim 1 wherein said substrate comprises glucose and adenosine tri-phosphate (ATP).

3. The method of claim 1 wherein said GK reaction has a higher maximal activation than observed in a coupled GK assay.

4. The method of claim 1 further comprising steps for assaying for modulators of GK wherein
said incubating step a. further comprises:
i. incubating said GK with substrate in a control reaction vessel; and
ii. incubating said GK with substrate and a test compound in a test reaction vessel;
said initiating step b. further comprises initiating a GK reaction in each reaction vessel; and
said determining step d. further comprises determining ADP concentration in each reaction vessel using HPLC; and comparing ADP concentration from said control and test reaction vessels, wherein a higher ADP concentration in said test reaction vessel, relative to said control reaction vessel, reflects that said test compound is a GK activator.

5. The method of claim 4 wherein said substrate comprises glucose and ATP.

6. A method for measuring GK activity comprising:
a. incubating GK with substrate and reagents, wherein said reagents comprise radiolabeled glucose;
b. initiating a GK reaction to yield reaction products;
c. stopping said reaction;
d. filtering said reaction products;
e. washing said reaction products;
f. eluting said reaction products; and
g. determining glucose-6-phosphate concentration by quantification of radiolabeled glucose-6-phosphate produced by said GK reaction.

7. The method of claim 6 wherein said radiolabeled glucose is tritiated glucose.

8. The method of claim 6 wherein said reagents comprise glucose in a range of about 0.33 mM to about 50 mM.

9. A method for screening for modulators of GK comprising:
a. incubating GK with substrate and reagents, wherein said reagents comprise radiolabeled glucose, in a control reaction vessel;
b. incubating GK with substrate, reagents, and a test compound, wherein said reagents comprise radiolabeled glucose, in a test reaction vessel;
c. initiating a GK reaction to yield reaction products in each vessel;
d. stopping said reaction;
e. filtering said reaction products;
f. washing said reaction substrates and reagents;
g. eluting said reaction products;
h. determining glucose-6-phosphate concentration by quantification of radiolabeled glucose-6-phosphate produced by said GK reaction; and
i. comparing glucose-6-phosphate concentration from said control and test reaction vessels, wherein a higher glucose-6-phosphate concentration in said test reaction vessel, relative to said control reaction vessel, reflects that said test compound is a GK activator.

10. The method of claim 9 wherein said method is conducted in a high-throughput format.

11. The method of claim 9 wherein said radiolabeled glucose is tritiated glucose.

12. The method of claim 9 wherein said reagents comprise glucose in a range of about 0.33 mM to about 50 mM.

13. The method of claim 9 wherein said reaction is initiated by addition of Mg-ATP.

14. The method of claim 9 wherein said reaction is stopped by addition of formic acid.

15. The method of claim 9 wherein said reaction substrates and reagents are washed with water and said reaction products are eluted with ammoniumformate.

16. A method for measuring GK activity comprising:
a. incubating GK with glucose in a first reaction vessel;
b. adding Mg-ATP to said first reaction vessel to initiate a GK enzymatic reaction and thereby form a reaction mixture;
c. stopping said GK enzymatic reaction;
d. transferring said reaction mixture to a second reaction vessel;
e. adding Thio-NAD and glucose-6-phosphate dehydrogenase to said reaction mixture in said second reaction vessel;
f. mixing the contents of said second reaction vessel; and
g. determining glucose-6-phosphate concentration by measurement of the absorbance of contents of said second reaction vessel and correlating absorbance values to a glucose-6-phosphate standard curve.

17. The method of claim 16 wherein said reaction is carried out using a multi-well microtiter plate.

18. The method of claim 16 wherein said glucose is serially diluted over a range of concentrations from 0 to about 50 mM.

19. The method of claim 16 wherein said first reaction vessel is cooled on ice prior to transfer to said second reaction vessel.

20. The method of claim 16 wherein said reaction is stopped using heat or EDTA.

21. A method for screening for modulators of GK activity comprising:
a. incubating GK with glucose in a first control reaction vessel;
b. incubating GK with glucose and a test compound in a first test reaction vessel;
c. adding Mg-ATP to said first control and test reaction vessels to initiate GK enzymatic reactions and thereby form reaction mixtures;
d. stopping said GK enzymatic reactions;
e. transferring aliquots of said reaction mixtures to a second control and second test reaction vessel;
f. adding Thio-NAD and glucose-6-phosphate dehydrogenase to said reaction mixture in said second control and test reaction vessels;
g. mixing the contents of said second control and test reaction vessels;
h. determining glucose-6-phosphate concentration by measurement of the absorbance of the contents of said second control and test reaction vessels;
i. correlating absorbance values obtained from said second control and test reaction vessels, to a glucose-6-phosphate standard curve; and
j. comparing glucose-6-phosphate concentration from said second control and test reaction vessels, wherein a higher glucose-6-phosphate concentration in said second test reaction vessel, relative to said second control reaction vessel, reflects that said test compound is a GK activator.

22. The method of claim 21 wherein said reactions are carried out using multi-well microtiter plates.

23. The method of claim 21 wherein said glucose is serially diluted over a range of concentrations from 0 to about 50 mM.

24. The method of claim 21 wherein said first control and test reaction vessels are cooled on ice prior to transfer to said second control and test reaction vessels.

25. The method of claim 21 wherein said reaction is stopped using heat or EDTA.
